(19) United States
(12) Patent Application Publication
(10) Pub. No.: US 2018/0282169 A1
(43) Pub. Date: Oct. 4, 2018

(54) SILICA SYNTHESIS

(71) Applicant: UNIVERSITY OF SHEFFIELD, SHEFFIELD (GB)

(72) Inventors: JOSEPH RICHARD HENRY MANNING, SHEFFIELD (GB); SIDDHARTH VIJAY PATWARDHAN, SHEFFIELD (GB)

(21) Appl. No.: 15/757,182

(22) PCT Filed: Sep. 1, 2016
(86) PCT No.: PCT/GB2016/052705

§ 371 Date: Mar. 2, 2018

(30) Foreign Application Priority Data
Sep. 3, 2015 (GB) 1515644.1

(51) Int. Cl. C01B 33/12; B01J 39/04; B01J 39/18; C07C 209/86

(52) U.S. Cl. CPC C01B 33/126; C07C 209/86; B01J 39/18; B01J 39/04

(57) ABSTRACT

A method for the production of a silica includes (1) reacting a silicic acid solution with an amine, to precipitate the silica from an aqueous solvent system. The method also includes removing at least a portion of the amine associated with the precipitated silica by treating the silica in an aqueous solvent system at a pH that is reduced from that of the reaction mixture (2). The reduction in pH may be by first reducing the pH of the aqueous solvent system, and then removing the silica from the aqueous solvent system. Alternatively the reduction in pH may be removing the silica from the aqueous solvent system first, before treating the silica with an aqueous solvent at a pH below that of the silica forming reaction. Subsequently the silica is removed from the solution (3).

### FIELD OF THE INVENTION

The present invention relates to methods for manufacturing silica and methods for adjusting the properties of the product, including purification.

### BACKGROUND TO THE INVENTION

The term bioinspired silica refers to silica produced by techniques that mimic to at least some extent the production of silica (biomineralisation) found in the natural, especially the marine world. Typically bioinspired silicas are formed in the presence of a template material such as those found in natural biological silica formation or synthetic analogues of such materials. Examples include polypeptides, polysaccharides, synthetic polymers or small molecules such as amines or polyfunctional amines.

The template materials, also described as additives, act as a structure-directing agent to aid in silica formation. Typical amines employed are linear or branched analogue of ethylene diamine, although a wide range of alternatives have been reported [1]. The activity of these additives have been widely investigated, and it is believed that they are important in all aspects of the synthesis, from catalysing the initial condensation of silicic acid monomers, to promoting aggregation of colloidal silica oligomers, to controlling the macrostructure of the precipitated silica particles.

The originally formed bioinspired silica particles are a composite of the inorganic silica material and some occluded organic template material. Typically the template material is removed by a calcination (heating to high temperature) procedure.

The otherwise mild synthesis of bioinspired silica opens up the possibility of using bioinspired synthesis to include encapsulation of bioorganic species such as enzymes and even whole cells offering protection and even improved activity over the bare biomaterials. This strategy of directly functionalising the silica during its synthesis represents a great improvement over two-step functionalisation methods of calcination and chemical tethering which is the common functionalisation method for all porous silicates.

The opportunity to manufacture silica with controlled structure and porosity offered by bioinspired techniques together with the potential for using the silica products as carriers for a wide range of materials including bioorganic species provides the need for improved methods and techniques in silica manufacture.

Bioinspired silica products may find use, for example, as catalysts, sorbents, fillers, excipients/additives food and drug additives, and for molecular storage.

### DESCRIPTION OF THE INVENTION

The present invention provides a method for the production of a silica, the method comprising:
reacting a silicic acid solution with an amine, to precipitate the silica from an aqueous solvent system; and
removing at least a portion of the amine associated with the precipitated silica by treating the silica in an aqueous solvent system at a pH that is reduced from that of the reaction mixture.

Typically the silica is formed at a pH of about 7, in the range of from 5 to 9, or even 6.5 to 7.5. A pH of 6.5 to 7.5 has been found to provide larger particles, that precipitate more easily and in a shorter timescale. The removal of at least a portion of the amine associated with the silica is carried out at a pH below that of the silica forming reaction.

Thus the present invention provides a method for the production of a silica, the method comprising:
reacting a silicic acid solution with an amine, to precipitate the silica from an aqueous solvent system; and either:
   a) reducing the pH of the solvent system; and then
   b) removing the silica from the aqueous solvent system; or
   a) removing the silica from the aqueous solvent system; and then
   b) treating the silica with an aqueous solvent at a pH below that of the silica forming reaction and subsequently removing the silica from the solution.

Surprisingly the use of tap water rather than specially purified water has been found not to affect the quality of the product produced by the processes described herein. Without being bound by theory it appears that the formation of the silica is controlled by the presence of additives rather than homogeneous nucleation or colloidal stability per se. The use of the amine additives and the resulting organic-inorganic interactions appear less sensitive to ionic strength than other templated silica systems. Thus the methods described herein do not appear sensitive to the impurities typically present in potable water and so the process conditions and product properties are not adversely affected.

The silicic acid may be prepared in any conventional way, for example from sodium silicate and a mineral acid, such as hydrochloric acid or sulphuric acid; or from other silicic acid sources such as hydrolysis of TEOS (tetraethyl orthosilicate). Other possible silica precursors include—alkoxy silanes (e.g. TMOS—tetramethyl orthosilicate), diol-modified silanes such as tetrakis(2-hydroxyethoxy)silane [Ref 4], organic complexes of silicon (e.g. hexavalent catechol complex), silica sol (suspended silica nanoparticles [Ref 5]), and those derived from biology (e.g. from rice hulls [Ref 6] or diatoms in the form of diatomaceous earth). Industrial waste streams may also be employed as a source of silica [Ref 7]. Industrial waste streams such as cement kiln dust, construction and demolition waste, steel making slag or residual combustion waste [ref 11] may be employed as inexpensive source of silica.

The reaction to produce the silica is typically carried out at a pH of about 7, for example from pH 5 to 9 or even from pH 6.5 to 7.5. Agitation, for example in the form of stirring is normally employed to ensure good mixing.

The reduction of the solvent system pH (typically to <7) or the treatment with an aqueous solvent at a more acid pH (typically a pH of <7) can be conveniently done with a mineral acid such as hydrochloric acid or sulphuric acid or by forming an acidic system e.g. hydrogen chloride gas dissolving in the aqueous system. Other acids may be employed such as HNO₃, HBr, HF, H₃PO₄ and H₃BO₃. Acids such as H₃PO₄ and H₃BO₃ have a buffering effect which can alter the morphology of the silica product produced [Refs 8, 9].

Other sources of acidity, such as organic acids may be employed. Examples include acetic acid, formic acid and citric acid.

Mixtures of acids may be employed.

The initial reaction to produce a silica will produce a silica “templated” by the amine i.e. the initially formed silica has amine associated with it. The amine may be bound to the silica by ionic and/or hydrogen bonding and the amine may reside in pores formed in the silica structure. The silica products formed by the methods described herein are porous, with the structure and porosity determined by the reaction conditions and especially the amine “additive” employed in the reaction mixture.

In contrast to prior art methods of silica synthesis employing amine containing “additives” to direct the silica structure formed, the method described herein provides a convenient means of controlling the amount of additive remaining in the silica. The treatment at a more acidic pH can be adjusted to remove a desired proportion of the amine or even all or substantially all of the amine. As described further hereafter by means of example, the lower the pH, the greater the removal of the amine additive from the precipitated silica, up to and including complete removal or substantially complete removal of the amine additive.

The range of pH employed to remove the amine additive may be for example from pH 8 to 1, for example pH 6 to pH 1, or even from pH 2 to pH 4. For complete or substantially complete removal of the amine additive a pH treatment at low pH is employed. For example, from pH 3 to pH 1 or lower. A pH of about 2 is typically effective in removal of all or substantially all of the amine additive.

Thus the methods described herein allow the production of a wide range of silica, with structure and porosity determined by the reaction conditions and the additive composition employed. The post formation treatment at a lower pH then allows a deliberate choice of additive content, or its removal under mild conditions. This contrasts with prior art methods where additive removal is carried out by making use of a calcination step that is expensive, is destructive to the additive material employed, and can degrade the detailed structure of the silica from that originally produced.

A wide range of amines may be employed. Amines having at least one H present on the nitrogen may be employed. Thus the method can be operated with primary, secondary or tertiary amines. Acid salts of such amines may be employed as the source of the amine, provided the pH of the reaction mixture—silicic acid solution with the amine—is adjusted to that appropriate for producing a silica. Polyfunctional amines may be employed. Generally the amine, or acid salt of the amine, employed has at least one H present on the nitrogen, as quaternary systems of the form (R)₄N X⁻, where none of the groups R are —H, are not readily displaced from their association with the silica by the methods described herein.

Studies indicate that the removal of the amine from the silica proceeds via the increasing protonation of the amine to the quaternary form and corresponding decreases in negative charge on the silica surface i.e. change from siloxide Si—O⁻ to silanol Si—OH. This process reduces ionic bonding from amine to silica surface and, as pH is reduced further, results in loss of available hydrogen bond acceptors. Thus by adjusting the pH treatment, the amine silica interactions reduce in strength and in number, allowing the amine to diffuse away from the silica surfaces.

Suitable amines include polyamines (amines having at least two amine functions) including at least one N—H group. TETA (triethylentetramine) or PEHA (pentaethylenehexamine) are examples. The polymers poly(ethyleneimine) (PEI) and poly(allyl amine) (PAA) are effective for silica formation for a range of polymer weights. Generally for a polymeric amine as the molecular weight of polymers increases, the effect of post-synthetic modification changes, producing larger pores or sometimes no pores at all. With higher molecular weight amines the removal step, by reducing the pH may be less effective.

Further examples of suitable amines are bis(3-aminopropyl)amine (containing both primary and secondary amine groups), and bis(3-dimethylaminopropyl)amine (containing secondary and tertiary amine groups) [Ref 10]. Naturally occurring polyamines such as spermidine and spermine may also be employed.

Mixtures of amines may be employed.

Following the adjustment of the amine additive content in the further processing is employed, usually to result in a dried product. Thus the method can include further process steps such as washing the silica with aqueous and/or non-aqueous solvent and drying the product. As the amine additive content has been adjusted to the desired level by the pH<7 treatment, drying can be mild in comparison to the high temperature calcining step normally employed where a silica with little or no additive content is required.

Where the silica formed is collected by removing from a solution or solvent system this is conveniently done by filtration in the conventional manner. However, decanting, removal of the solution or solvent system from above the settled silica solids, may also be employed and may be convenient for some process steps, especially at a large scale.

The methods of the present invention may be operated as conventional batch processes. The reduction in pH step to remove the amine additive allows a fine control of the amount of amine left in the dried silica product. As calcination is not required, even when producing an amine free or substantially amine free silica, reduced energy costs and process time can be achieved.

Further advantages can be obtained by recycling the reduced pH aqueous solvent stream after the silica has been removed from it. The reduced pH aqueous solvent stream typically comprises the amine additive or additives, acid or acids and, depending on the source of silicic acid, salts such as sodium salts of the acid (originating from e.g. sodium silicate as input source of silicic acid). Typically mostly or all of the amine or amines are in in the form of salts of the acid or acids present, from the acid used in the initial reaction to form the silica and also as subsequently added to reduce the pH.

Recycling the reduced pH aqueous solvent stream allows the amine additive to be used repeatedly in the formation of batches of silica; it is not lost by degradation as with processes making use of calcination. This provides a substantial saving in both water usage and amine usage. It also avoids waste disposal costs from effluent streams where the amine containing stream is only used once. To reuse the stream it is returned to the reaction vessel used for forming the silica and the appropriate quantities of silicic acid source, and further water and/or other solvent are added to obtain a mixture for reaction to produce more silica. Adjustment of pH by adding more acid or adding more amine, or even an auxiliary base may be carried out as required.

Where a build-up of salts with repeated reuse of the reduced pH aqueous solvent stream is interfering with the silica manufacture, such salts can be removed by suitable processes. For example where sodium silicate is used as a source of the silicic acid and hydrochloric acid is employed an accumulation of sodium chloride in the reduced pH liquid stream will occur. Any unwanted effects of this build-up of sodium chloride may be alleviated by dilution and/or periodic disposal of the reduced pH stream or at least a portion of it. If desired the salts build up may be addressed by use of ion exchange resins. A cation exchange resin may be used to remove sodium ions in this example.

Advantageously the process of the invention may be operated as a continuous process. This has a number of economic advantages in terms of improved output of silica per hour. Large scale production of a silica can readily be contemplated by means of the continuous process described herein.

A typical continuous process may comprise a continuous or substantially continuous feed of silicic acid precursor, amine additive and acid to a reaction vessel, where the conditions and residence time are sufficient to produce a silica. The product stream, silica with amine additive as template, flows out to a second vessel where more acid is added to reduce the pH. The acidified mixture flows out of the second vessel to a solids collection system, typically a filter or filters. Washing and then drying steps finish the product to a dried silica with the desired amine content, even a silica with no or substantially no amine content. Conveniently the solids collection is carried out in a two-step process such as filtration or decantation in a solids collection unit to remove the process liquid (reduced pH aqueous solvent stream) followed by transfer to a washing unit where the silica is washed (e.g. by water on a filter or filters, before drying). This procedure avoids diluting the reduced pH stream with wash solvent (usually water). Alternative convenient arrangements for solids collection and washing can include decanting centrifuges or continuous belt type filters. Such arrangements also allow for separation of the original reduced pH liquid stream or at least the bulk of it, from the wash solvent.

The continuous process may conveniently include recycle of the reduced pH aqueous solvent stream after the silica has been removed from it. This can be carried out in a manner analogous to that discussed above with respect to the batch process. After removal of the precipitated silica the stream is recycled to the first reactor, where the silica is formed, and the other inputs to the process are adjusted to allow for the returning amine content and to make the required adjustment to the pH. If there is a build-up of salts with a recycle of the reduced pH stream, then the procedures as discussed above with respect to batch processing may be employed, including the use of ion exchange resins to remove cations.

### BRIEF DESCRIPTION OF THE DRAWINGS

FIG. 1 shows graphically the nitrogen concentration by weight in a silica composite with respect to the degree of acidification;

FIG. 2 shows graphically porosity of silica samples against the pH of an acid treatment;

FIGS. 3a and 3b show graphically surface area measurements of silica against pH; and

FIGS. 4, 5, 6 and 7 show schematically processes for manufacturing silica.

### DESCRIPTION OF SOME EMBODIMENTS BY WAY OF EXAMPLE

### Experiments in Silica Synthesis

In a typical procedure silica was synthesised by mixing solutions of sodium silicate pentahydrate and pentaethylenehexamine (PEHA) such that their final concentrations were 30 mM and 5 mM respectively (corresponding to a 1:1 ratio of silicon to nitrogen). This mixture was subsequently neutralised using 1M HCl, (pH 7.0±0.05) and allowed to react under mixing for 5 minutes.

Once the reaction was complete, the mixture was separated into aliquots, each being acidified until the appropriate pH had been reached. Acidification was carried out after the reaction was complete by gradual addition of 1M HCl under manual mixing until the reaction mixture had reached the desired pH.

Finally the silica particles were isolated by centrifugation for 15 minutes at 8000 rpm repeated three times, and dried in an oven at 85° C. overnight. Between each repeat, the effluent water was decanted and replaced by pure water to serve as washing. The silica cake was re-suspended by manual shaking before returning to the centrifuge.

The silica synthesised was analysed initially using CHN elemental analysis and nitrogen adsorption, on a Perkin Elmer 2400 Series II CHNS Analyser and a Micromeritics ASAP 2420, respectively. Further analysis of the silica samples was carried out using carbon dioxide adsorption in a Hiden Isochema Intelligent Gravimetric Analyser (IGA).

### Post-Reaction Acidification in the PEHA-Silica System

In this study, PEHA was initially used as a model compound for the investigation into template removal due to its catalytic activity towards silica formation. PEHA-silica was synthesised according to the established bioinspired method [2] after which the reaction mixture was partitioned and further titrated with HCl so that a range of pH values could be compared. This produced a series of PEHA-silica composites treated after the synthesis with environments ranging from pH 7 and pH 2 (the isoelectric point of silica).

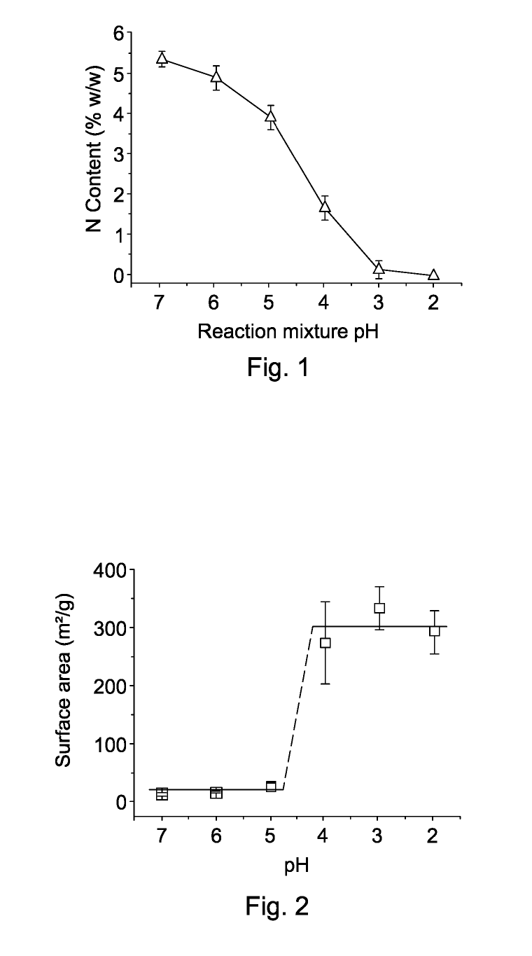

The amount of organic material present within the silica structures was determined using CHN elemental analysis. FIG. 1 shows the nitrogen concentration by weight in the bioinspired silica composite with respect to the degree of acidification, indicating the amount of template remaining within the material structure.

After acidification to pH 3 or lower, down to pH 2 the nitrogen concentration falls below the limit of detection of the method employed, indicating that all of the template has been removed from the structure. These results show that post reaction reduction of pH (acidification) can be used to fully remove the template material from bioinspired silica.

The porosity of the samples, as measured by surface area, was also examined and the results are shown graphically in FIG. 2.

The majority of the change takes place between pH 5 and pH 4 with no major porosity at higher pH, and no clear trend in the porosity at lower pH. It is worth noting that while there appears to be a maximum at pH 3, the three values from pH 4 to pH 2 are not significantly different (p>0.23 in all cases).

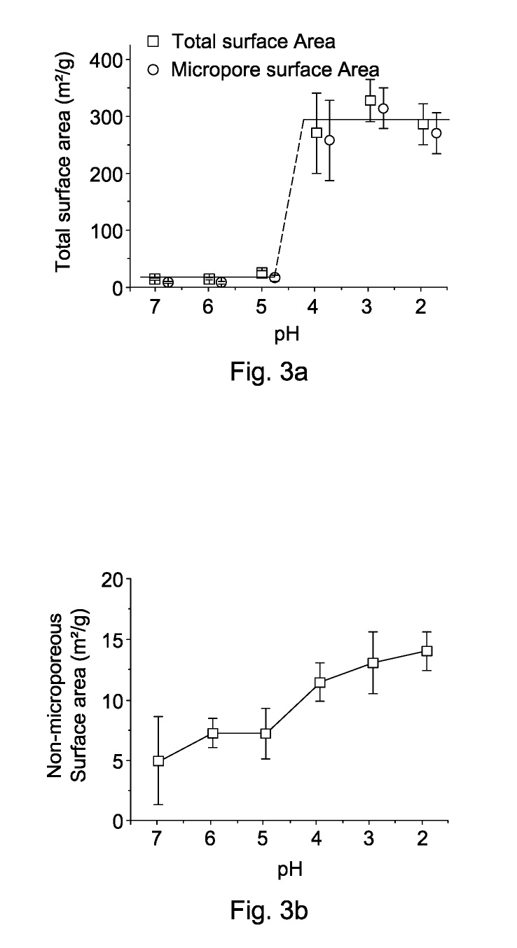

These surface area measurements can be complemented by using the t-plot method developed by de Boer [Ref 3] to estimate the difference between micropores and larger pores in the material. These are compared to the overall surface area in FIG. 3a, with the external surface area being shown in FIG. 3b.

From this, it can be seen that the majority of the pores generated in the post-treatment are in the microporous region in these examples. This suggests that the acidification treatment removes the amine from the micropores in the silica. There is a very slight increase in the larger surface area (FIG. 3b), which may also be due to the amine removal, but due to the small size of the template molecule PEHA this cannot be easily concluded.

Similar processing to that described above may also be employed to produce mesoporous silica, typically by changing the amine template employed.

### Use of Functionalised Silica

The PEHA-silica system may be used for CO₂ absorption, making use of amine content, deliberately left associated with the silica.

### Process Outlines

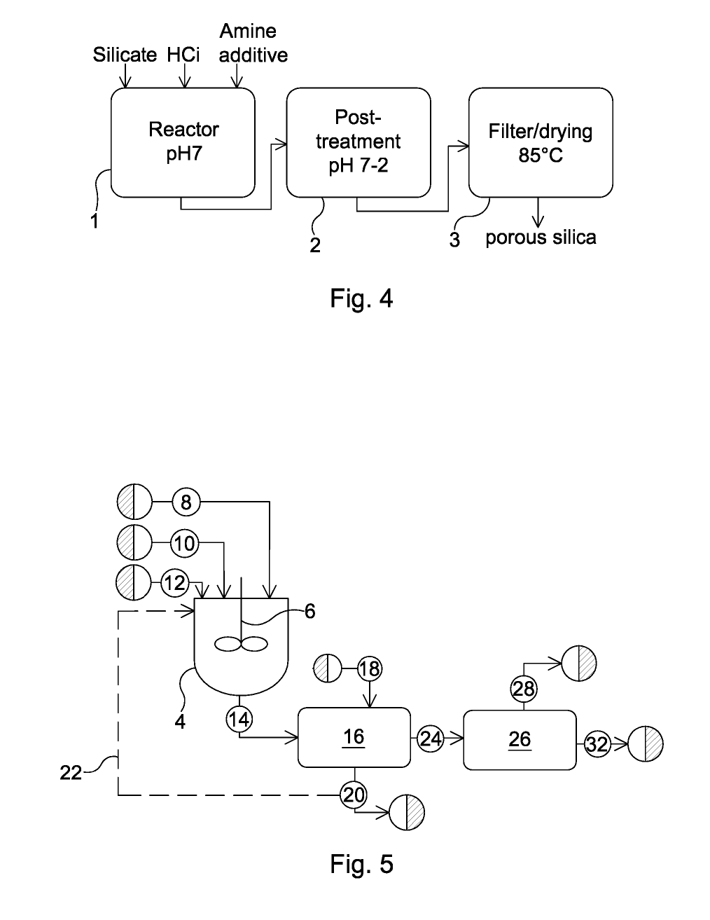

In FIG. 4 a schematic flow chart showing the general approach of the method is depicted. In this example a silicate (such as sodium silicate) is reacted with hydrochloric acid in water to provide silicic acid. An amine additive provides the template. In step 1 the mixture is stirred in the reactor at a about a pH of 7 for example between pH 9 and pH5, more typically pH 6.5 to 7.5 to produce the silica solids, associated with the amine acting as a template. A typical temperature range for reaction is mild, for example 15° C. to 35° C. The concentration range of the silicon is typically from 20-100 mM. The pH of the system is then adjusted (reduced) in step 2 to release the desired proportion of amine from the silica solids. A pH as low as 2 can completely release the amine as discussed above.

Filtering, washing (with water, aqueous solvent or solvent) and then drying of the silica (step 3) produces the porous silica product, with a desired amine content, including the option of no or substantially no amine content may be obtained. Drying can be carried out at moderate temperature, say from 70° C.-120° C., for example 85° C. as indicated in the figure. Vacuum or spray drying may be employed. Drying at moderate temperatures allows retention of amine in the product, when desired. Conversely there is no need to employ high temperatures (e.g calcining at say 500° C.) if complete amine removal is required.

### Batch Processing

FIG. 5 illustrates schematically, by way of example, the process of FIG. 4 showing the main process equipment employed. Reactor 4, equipped with agitator 6 has connection to input supplies of acid 8, amine 10 and silicate 12. Other possible inputs include a solvent source (typically water). After addition of acid, silicate, amine and water the reaction is carried out with agitation to produce the bioinspired silica.

The reaction mixture is then passed via outlet 14 to filter 16. Filtering is carried out in the usual way with wash supply 18 used to remove the bulk of remaining traces of the reaction mixture fluid from the solids. The fluid effluent 20 from the filter 16 may be disposed of. Alternatively, as suggested by dashed line 22 at least a portion may be recycled to reactor 4, to supply amine for the next batch, with pH adjusted to the reaction pH (7) by use of less acid from supply 8 than for a batch making use only of unrecycled materials.

After filtering and washing the silica solids are passed 24 to drier 26. When dried by driving off the residual water 28 the finished silica product is removed 32.

### Continuous Processing

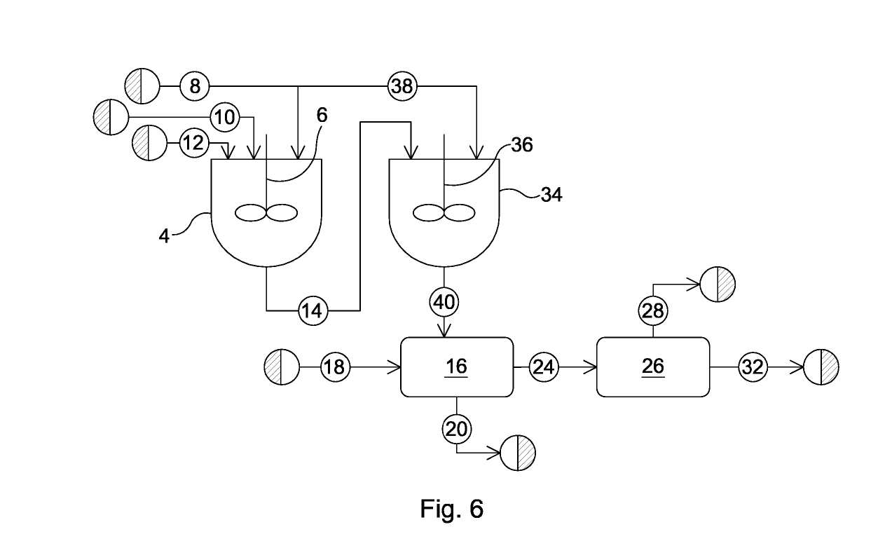

FIG. 6 illustrates schematically, by way of example, a continuous process in accordance with the methods described herein. Like numbered parts are numbered the same as in FIG. 6. Reactor 4 has connection to input supplies of acid 8, amine 10 and silicate 12. In operation the reactor 5 is arranged so that reaction mixture flows continuously out of outlet 14 to a second reactor 34, equipped with agitator 36. Input supplies of acid 8, amine 10 and silicate 12 are adjusted to maintain the pH and keep reactor 4 supplied with sufficient material for continuous operation of the process.

In reactor 34 acid supply 38 is used to reduce pH to the desired level in the reaction mixture flowing from reactor 4 via outlet 14. The acidified reaction mixture flows out of reactor 34 via outlet 40 to filtration unit 16 where filtering and washing (from supply 18) is carried out, with the washed and filtered solids progressing to drier 26.

Continuous Processing with Recycle of Amine

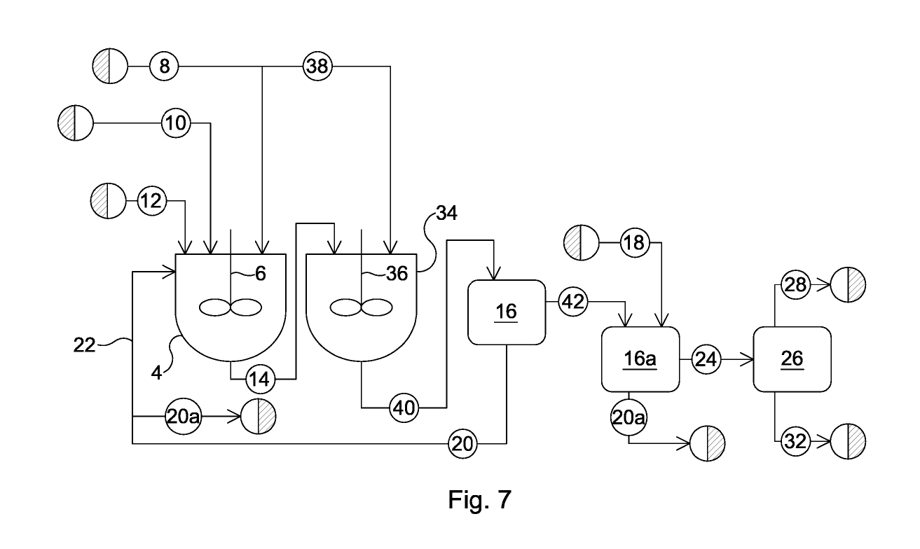

FIG. 7 illustrates schematically, by way of example, a continuous process in accordance with the methods described herein. Like numbered parts are numbered the same as in FIGS. 4 and 5.

The process is operated in a similar manner to that described in FIG. 6 except that there is a recycle of reaction mixture fluid and, in this example, filtering silica solids from the reaction mixture fluid and subsequent washing of the solids is carried out in separate units.

After production of silica in reactor 4 followed by reduction of pH in reactor 34 the reaction mixture flows via outlet 40 to filter 16, where the solids are separated from the reaction mixture fluid. The fluid (which will contain any silica particulates that have passed through the filtration medium in filter 16) leaves the filter via outlet 20 and is recycled into reactor 4 (line 22), with the option of removal of a portion of the effluent via line 20a, to adjust process volume.

The wet solids from filter 16 are passed 42 to wash filter 16a, where they are washed from wash supply 18 resulting in a wash effluent exiting from outlet 20a. Thus the wash employed is kept separate from the reaction mixture fluid, avoiding dilution of the recycled material and allowing the option of using different wash fluids in the process. An alternative means of achieving this would be, for example, to make use of a continuous belt filter with wash fluid supplied towards the end of the belt, after the bulk of the reaction mixture fluid had been filtered off and sent for recycle.

Finally, the washed solids then pass 24 to drier 26.

Study of a typical continuous process, such as that described above, shows that if a recycle of ˜90% by volume of the filtered off reaction mixture fluid is carried out, then the process is significantly intensified, in comparison with a process without recycle. The yield of silica may increase from ˜1 g/l to ˜10 g/l of reaction mixture with a requirement for amine template reducing from ˜1 kg amine per kg of silica produced to ˜0.04 kg amine per kg of silica produced (assuming a silica free of amine content is being made.)

### REFERENCES

The entire content of each of these documents is incorporated by reference herein.
1.
a. Patwardhan, S. V. Biomimetic and bioinspired silica: recent developments and applications, Chem. Commun., 2011, 47(27), 7567-7582.
b. WO2010036344, Miller et al., 2010, Compositions, oral care products and methods of making and using the same.
c. U.S. Pat. No. 6,670,438, Morse, et al., 2003, Methods, compositions, and biomimetic catalysts for in vitro synthesis of silica, polysilsequioxane, polysiloxane, and polymetallo-oxanes.
d. U.S. Pat. No. 7,335,717, Morse, et al., 2008, Methods, compositions, and biomimetic catalysts for the synthesis of silica, polysilsequioxanes, polysiloxanes, non-silicon metalloid-oxygen networks, polymetallo-oxanes, and their organic or hydrido conjugates and derivatives.
2. C. Forsyth and S. V Patwardhan, “Controlling performance of lipase immobilised on bioinspired silica,” J. Mater. Chem. B, vol. 1, no. 8, pp. 1164-1174, 2013.
3. J. H. de Boer, B. C. Lippens, B. G. Linsen, J. C. P. Broekhoff, A. van den Heuvel, and T. J. Osinga, “The t-curve of multimolecular N₂-adsorption,” J. Colloid Interface Sci., vol. 21, no. 4, pp. 405-414, 1966.
4. Patwardhan S. V., Raab C, Husing N. and Clarson S. J., Macromolecule Mediated Bioinspired Silica Synthesis Using A Diol-Modified Silane Precursor, Silicon Chemistry, 2003, 2(5-6), 279-285.
5. Coradin, T.; Durupthy, O.; Livage, J. Langmuir 2002, 18, 2331.
6. Asuncion, M, Hasegawa, I, Kampf, J, and Laine, R. The selective dissolution of rice hull ash to form [OSiO1.5]8[R4N]8 (R=Me, CH2CH2OH) octasilicates. Basic nanobuilding blocks and possible models of intermediates formed during biosilification processes, Materials Chemistry, 2005, 15, 2114-21.
7.] Dodson, J R; Cooper, E C; Hunt, A J; Matharu, A; Cole, J; Minihan, A; Clark, J H; Macquarrie, D J, GREEN CHEMISTRY 2013, 15(5), 1203-1210.
8. Jantschke A., Spinde K., and Brunner E., Electrostatic interplay: The interaction triangle of polyamines, silicic acid, and phosphate studied through turbidity measurements, silicomolybdic acid test, and 29Si NMR spectroscopy, Belstein J. Nanotechnol., 2014, 5, 2026-2035.
9. Mizutani T., Nagase H., Fujiwara N., and Ogoshi H., Silicic Acid Polymerisation Catalyzed by Amines and Polyamines, Bull. Chem. Soc. Jpn., 1998, 71, 2017-2022.
10. Belton D. J., Patwardhan S. V., Annekov V. V., Danilovtseva E. N., and Perry C. C., From biosilicification to tailored materials: Optimizing hydrophobic domains and resistance to protonation of polyamines, PNAS, 2008, 105(16), 5963-5968.
11. P. Renforth, C.-L. Washbourne, J. Taylder, and D. A. C. Manning, Silicate Production and Availability for Mineral Carbonation, Environmental Science & Technology 2011, 45(6), 2035-2041.

1. A method for the production of a silica, the method comprising:
reacting a silicic acid solution with an amine, to precipitate the silica from an aqueous solvent system; and
removing at least a portion of the amine associated with the precipitated silica by treating the silica in an aqueous solvent system at a pH that is reduced from that of the reaction mixture.

2. The method of claim 1 wherein the pH of reaction to precipitate the silica is between pH 9 and pH 5.

3. The method of claim 1 wherein the pH of reaction to precipitate the silica is between pH 7.5 and pH 6.5.

4. The method of claim 1, wherein the step of treating the silica in an aqueous solvent system at a pH that is reduced from that of the reaction mixture, is carried out by reducing the pH of the reaction mixture aqueous solvent system; and then removing the silica from the said reaction mixture aqueous solvent system.

5. The method of claim 1, wherein the step of treating the silica in an aqueous solvent system at a pH that is reduced from that of the reaction mixture, is carried out by: removing the silica from the reaction mixture aqueous solvent system; and then treating the silica with an aqueous solvent at a pH below that of the silica forming reaction; and subsequently removing the silica from the solution.

6. The method of claim 1, wherein the silicic acid is prepared from at least one of sodium silicate, tetraethyl orthosilicate, alkoxy silanes, diol-modified silanes, organic complexes of silicon, silica sol, rice hulls, cement kiln dust, silica containing wastes from construction and demolition, steel making slag and residual combustion waste containing silica.

7. The method of claim 1, wherein the step of removing at least a portion of the amine associated with the precipitated silica is accomplished by the use of an acid selected from the group consisting of HCl, H₂SO₄, HNO₃, HBr, HF, H₃PO₄ H₃BO₃, acetic acid, formic acid, citric acid and mixtures thereof.

8. The method of claim 1, wherein substantially all of the amine is removed from the silica.

9. The method of claim 1, wherein the step of removing at least a portion of the amine associated with the precipitated silica is carried out at a pH of from pH 8 to pH 1.

10. The method of claim 9 wherein the step of removing at least a portion of the amine associated with the precipitated silica is carried out at a pH of from pH 6 to pH 1.

11. The method of claim 1, wherein the amine employed is a primary, secondary or tertiary amine, or an acid salt of the amine, and has at least one H present on the nitrogen.

12. The method of claim 11 wherein the amine or amine salt employed has at least two amine functions.

13. The method of claim 12 wherein the amine is selected from the group consisting of TETA (triethylentetramine), PEHA (pentaethylenehexamine), poly(ethyleneimine) (PEI), poly(allyl amine) (PAA), bis(3-aminopropyl)amine, bis(3-dimethylaminopropyl)amine, spermidine and spermine.

14. The method of claim 1, further comprising collecting the precipitated silica, washing and drying it.

15. The method of claim 1, wherein the production of the silica is operated as a batch process.

16. The method of claim 1, wherein the production of the silica is operated as a continuous process.

17. (canceled)

18. The method of claim 1, wherein a reduced pH aqueous solvent stream, containing amine removed from the silica produced, is recycled for further reaction with silicic acid.

19. The method of claim 18 further comprising removing salts from the reduced pH aqueous solvent stream by use of ion exchange resins.

20. A method comprising:
feeding, continuously or substantially continuously, a silicic acid precursor, an amine additive, and an acid to a reaction vessel, where the conditions and residence time are sufficient to produce a silica;
transferring the contents of the reaction vessel to a second vessel and reducing the pH;
transferring the contents of the second vessel to a solids collection system, where the silica is separated from the reduced pH aqueous solvent system; and
washing and drying the separated silica to produce a dried silica with a desired amine content.
